(19) United States
(12) Patent Application Publication
(10) Pub. No.: US 2010/0185080 A1
(43) Pub. Date: Jul. 22, 2010

(54) MAGNETIC RESONANCE GUIDED CANCER TREATMENT SYSTEM

(76) Inventor: Gunnar Myhr, Oslo (NO)

Correspondence Address: BACON & THOMAS, PLLC, 625 SLATERS LANE, FOURTH FLOOR, ALEXANDRIA, VA, 22314-1176, US

(21) Appl. No.: 12/664,790

(22) PCT Filed: Jun. 13, 2008
(86) PCT No.: PCT/GB08/02059

§ 371 Date: Mar. 16, 2010

Related U.S. Application Data
(60) Provisional application No. 60/929,223, filed on Jun. 18, 2007.

(30) Foreign Application Priority Data
Jun. 15, 2007 (GB) 0711662.7

(51) Int. Cl. A61B 5/055; A61N 7/02

(52) U.S. Cl. 600/411

(57) ABSTRACT

A system and method are provided for treatment of cancer, comprising: a focusable energy source for targeting a region of interest in a human or animal body to achieve hyperthermia in the region of interest; and a magnetic resonance imaging unit arranged to monitor at least one physical parameter related to oxygenation level spatially in and around the region of interest. The physical parameters may be one or more of partial oxygen pressure (pO₂), temperature, carbon dioxide level (CO₂) and acidity (pH).

The invention relates to a method and system for treatment of cancer and for monitoring biophysical effects associated with the cancer treatment using magnetic resonance imaging.

Non-surgical methods of cancer treatment, e.g. ionizing radiation and drug therapies, do not represent general specificity for cancer cells. Ionizing radiation can achieve a degree of specificity due to directional effects, while for anticancer drugs it is the proliferation of cancer cells that makes them susceptible to such treatment. In this respect the shear composite of a solid tumor limits the therapeutic index by structurally obstructing the delivery and efficacy of both ionizing radiation and drugs.

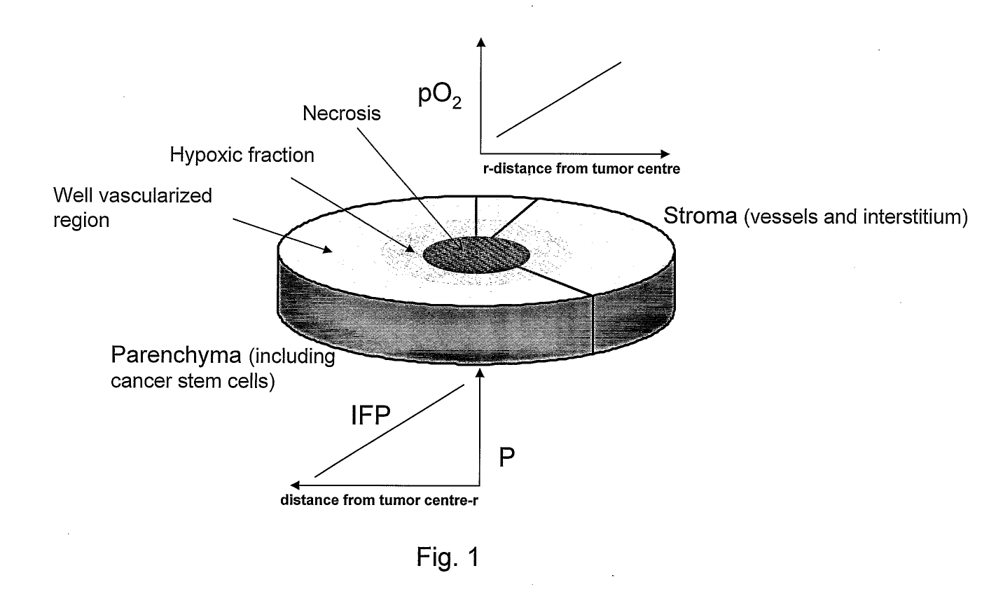

Molecules and particles cross vessel walls due to diffusion and convection. Diffusion is driven by osmosis or concentration gradients, while convection is driven by pressure gradients. The physiology of solid tumors has several characteristics that distinguishes them from normal tissues, which can cause the alteration of both diffusion and convection capabilities, and subsequently restrain the efficacy of cancer treatment, (see FIG. 1).

In healthy tissues the vascular system is balanced between proangiogenic and antiangiogenic molecules. Contrary to solid tumors, a lymphatic system is present to drain fluid and cellular byproducts from the interstitium. Normally, capillary pressure is in the 1-3 mm Hg range and the interstitial pressure is atmospheric. In contrast, tumor blood vessels are irregular, have arterio-venous shunts, blind ends, incomplete endothelial linings and basement membrane causing vessel leakage and demonstrating increased permeability to circulating particles and large molecules, enabling the Enhanced Permeability and Retention (EPR) effect. The break in the endothelium cause reduced hydrostatic pressure in the vessel and increased colloid osmotic pressure within the interstitium, which can elevate the Interstitial Fluid Pressure (IFP) by levels up to 100 mm Hg. IFP represent a barrier to drug delivery by decreasing transcapillary fluid flow and convective transport of compounds from the blood stream into the tumor interstitium. Impaired blood flow through the tumor also leads to reduced oxygen delivery. Normal tissue partial oxygen pressure (pO₂, also known as oxygen tension) ranges between 10 and 80 mm Hg, depending on tissue type, whereas tumors contain regions where pO₂ is less than 5 mm Hg. A cell that is low in oxygen responds by secreting cytokine. Tumor angiogenesis is found to be induced by a variety of pro-angiogenic cytokines, of which the best characterized is vascular endothelial growth factor (VEGF).

Tumor hypoxia is also of therapeutic concern because it can reduce the effectiveness of drugs and radiotherapy. By contrast, well oxygenated cells require one third of the dose of hypoxic cells to achieve a given level of cell killing.

The maximum oxygen diffusion distance in tumors is typically about 150 μm. Cell proliferation decreases as a function of the distance from the vasculature, as a result of reduced oxygen level. This can have knock-on effects for some chemotherapeutic agents which target cell mitosis.

Hypoxia and acidic pH (low extracellular pH) in tumors are primarily pathophysiologic consequences of the structurally and functionally disturbed vasculature and the deterioration of diffusion conditions. It is a characteristic feature of the imbalance between oxygen supply and demand, increased diffusion distances between the nutritive blood vessels and the tumor cells, and reduced O₂ transport capacity of the blood due to the presence of disease- or treatment-related anemia.

Partial oxygen pressure (pO₂) of human body fluids reflects the oxygenation or hypoxic status of the tissue in question. Previously, pO₂ measurements have been invasive, requiring either microelectrode/optode placement or fluid removal. In Zaharchuk G, et al, “Noninvasive oxygen partial pressure measurement of human body fluids in vivo using magnetic resonance imaging”, Acad. Radiol. 2006 Aug; 13(8):1016-24, an MRI based single-shot fast spin echo pulse sequence was modified, and longitudinal relaxation rate (R1=1/T1) was measured with a time-efficient nonequilibrium saturation recovery method which correlates with pO₂. This technique was originally developed to measure pO₂ in cerebrospinal fluid (CSF).

U.S. Pat. No. 5,397,562 describes a method of determining oxygen tension and temperature of tissue by administering a biologically compatible perfluorocarbon emulsion in an amount effective to generate a measurable NMR spectrum and comparing at least two spin-lattice relaxation rates measured in the ¹⁹F magnetic resonance spectrum to a predetermined relationship between spin-lattice relaxation rate and oxygen tension and temperature for the perfluorocarbon emulsion used.

Stem cells (SC) are characterized by their ability for self-renewal without loss of proliferation capacity with each cell division. Stem cells are immortal, and rather resistant to action of drugs. SC divide asymmetrically, producing two daughter cells, of which one is a new SC and the second is progenitor cell, which has the ability for differentiation and proliferation, but not the capability for self-renewal. Cancer stem cells (CSC) are in many aspects similar to SC. CSC make up as few as 1% of the cells in a tumor, making them difficult to detect and study. Like SC, CSC have a number of properties permitting them to survive traditional cancer chemotherapy and radiation therapy. These cells express high levels of ATP-binding cassette (ABC) drug transporters, providing for a level of resistance, are relatively quiescent, have higher levels of DNA repair and a lowered ability to enter apoptosis (some cancer treatments attempt to induce apoptosis). CSC might be the cause of tumor recurrence, sometimes many years after the appearance of the successful treatment of a primary tumor. Growth of metastases in distinct areas of body and their cellular heterogeneity might be a consequence of CSC differentiation and/or dedifferentiation and asymmetric division of CSC.

Aggressive new treatment approaches are desired to increase efficacy and improve the therapeutic index, not just towards the hypoxic fraction of solid tumors, but also towards CSC. With a more aggressive treatment, a greater number of CSC will be destroyed, reducing the tumor's ability to grow and to metastasize.

According to the invention, there is provided a system for treatment of cancer, comprising: a focusable energy source for targeting a region of interest in a human or animal body to achieve hyperthermia in the region of interest; and a magnetic resonance imaging unit arranged to monitor a physical parameter related to oxygenation level spatially in and around the region of interest.

The MR unit may be arranged to monitor partial oxygen pressure or it may be arranged to monitor carbon dioxide level which is related to partial oxygen pressure by a reciprocal function (i.e. f(1−pO₂)) or it may be arranged to monitor hyperthermia. Hyperthermia causes increased blood flow to the region of interest which brings with it more oxygen and is therefore an indication of oxygenation level. Or the MR unit may be arranged to monitor acidity (pH level) which is related to hyperthermia (hyperthermia reduces pH level) and is therefore also an indication of oxygenation.

According to the invention, there is provided a system for treatment of cancer, comprising: a focusable energy source for targeting a region of interest in a human or animal body to achieve hyperthermia in the region of interest; and a magnetic resonance imaging unit arranged to monitor at least one of partial oxygen pressure (pO₂), temperature, acidity (pH) and CO₂ level spatially in and around the region of interest.

The MRI unit may monitor absolute levels, relative levels or changes in levels of pO₂, temperature, and/or variations in other physical parameters like acidity (pH) and/or carbon dioxide (CO₂) (related to pO₂ as a function of (1−pO₂). More preferably, the MRI unit monitors these variables as a function of time.

By using an MRI machine to monitor temperature and/or pO₂, the quality control of the treatment is greatly increased. Temperature and pO₂ are both indicators of the degree of success of the treatment. Measurements of temperature indicate the direct result of the focused energy source, i.e. the level of hyperthermia in the region of interest. As tissue is heated, blood flow to the tissue is increased, thus bringing more oxygen into the tissue and correspondingly reducing tissue hypoxia. In other words, hyperthermia is the cause and increased pO₂ is the effect. Increased blood flow also increases transport of chemotherapeutic agents (e.g. liposomally encapsulated drugs) into the region of interest if these are used as part of the treatment. Although the increased pO₂ in the tissue is a consequence of the induced hyperthermia, the pO₂ level does not depend solely on temperature. It also depends on the physical structure (e.g. of the vasculature) of the tissue (e.g. tumor) in question. Measurements of pO₂ therefore give a further indication of the degree of success of the treatment by monitoring the combination of hypoxia and hyperthermia in the region of interest. Preferably both pO₂ and hyperthermia are. monitored. Measuring both cause and effect provides more information about the tissue in the region of interest.

Although the localized hyperthermia and consequent oxygenation of the region of interest can be sufficient treatment in themselves, preferably a further treatment modality is operatively connected to the magnetic resonance imaging unit. The further treatment modality may include, for example, any of: further hyperthermia for tissue ablation, a radiation unit (ionizing radiation) for radiotherapy, application of a chemotherapeutic agent for chemotherapy, application of ultrasound in order to activate a chemotherapeutic agent such as release of liposomally encapsulate drugs or application of ultrasound to induce cavitation of naturally occurring microbubbles in the region of interest. Combinations of these further treatment modalities may also be applied, e.g. chemoradiotherapy. If drugs are to be applied during the treatment, they are preferably multi-operable drugs, e.g. having dual capabilities as a radiation sensitizer as well as being cytotoxic.

The hyperthermia induced for increasing blood flow, and hence oxygenation and transport capability of the tissue, is typically only a few degrees centigrade, e.g. up to 40-43° C. in mammals. Such temperatures are not generally high enough to kill normal, healthy cells. Hyperthermia for tissue ablation generally requires higher temperatures, e.g. up to 55-80° C.

As discussed above, radiotherapy and chemotherapy are more effective (i.e. they induce more cell death) in well oxygenated tissue compared with hypoxic tissue. At the same time, the dose of radiation or chemicals which can be applied to a patient is limited by the toxicity of these treatments. Therefore, by monitoring pO₂ levels in the region of interest and using those measurements to control the chemotherapy or radiotherapy, the treatment dose can be specifically and accurately applied while the tissue is in the more receptive treatment state, thus increasing treatment effectiveness without increasing the level of toxicity to the patient, i.e. increasing the therapeutic index for the treatment. Preferably, treatment via the further treatment modality is begun when the partial oxygen pressure, carbon dioxide level and/or pH level reaches a threshold value.

The MRI machine can also be adapted to monitor cavitation levels within the region of interest. This may be in the form of monitoring acoustic streaming, stable and/or inertial (transient) cavitation. In the following the terms acoustic streaming, stable and/or inertial (transient) cavitation are collectively called or termed cavitation. Cavitation of naturally occurring or added microbubbles (e.g. liposomally or polymer encapsulated microbubbles) can increase the rate of uptake of chemotherapeutic agents administered to the region of interest. The effect of the cavitation can be calculated and therefore, by monitoring cavitation, the amount of drug uptake can be deduced. This data can be used simply as a quality control or it can be used as feedback for real time control of the treatment.

Low frequency ultrasound exposure can be used in combination with liposomally encapsulated chemotherapeutic agents. Such low frequency ultrasound treatment can be non-hyperthermic, but can significantly increase the effect of liposomally encapsulated cytostatic drugs on tumor growth. High frequency ultrasound exposure can be applied to the region of interest to induce hyperthermia for tissue ablation. A transducer unit can provide both low and high frequency ultrasound, simultaneously, concurrently or in sequence. The lower frequencies predominantly induce cavitation, while the higher frequencies are for inducing hyperthermia.

Real time MRI monitoring facilitates approximate real time concomitant treatment options, including various combinations of drug therapy, hyperthermia, ionizing radiation, ablation and other treatment options, before or after surgery, with optimization capabilities.

Using an MRI machine for monitoring enables spatial monitoring and mapping of the region of interest, i.e. providing maps or gradients of pO₂, temperature, pH and/or CO₂ in a region of interest. The MRI machine can also monitor as a function of time by taking repeated measurements.

By modelling the region of interest (e.g. a tumor) before treatment, i.e. spatially mapping tissue in the region of interest (which can be done via a variety of techniques including MRI and CT scans) and mapping the location of the region of interest with respect to reference points on the subject, it is possible to determine the levels of hyperthermia and pO₂, pH and/or CO₂ in relation to the position of the region of interest, i.e. the position within the body. This data can be used either to correct the focus of the energy source which is applying the hyperthermia (e.g. to maintain accurate targeting of the region of interest) and/or to control the directionality of the further treatment modality (e.g. to control the direction and/or focus of applied radiation and/or applied ultrasound) to maximise the treatment effectiveness. Other factors, such as timing, intensity, fractionation and overall treatment time, i.e. total energy applied, can also be calculated more accurately using modelling of the region of interest.

Further, by mapping and modelling the region surrounding the region of interest, the direction and focus of the energy source and/or the other treatment modalities can be selected so as to avoid obstacles such as bones and air pockets which could otherwise attenuate the energy and reduce treatment effectiveness. Navigation, guiding and tracking of the energy source and treatment modalities can be effected throughout the duration of the treatment.

Passive MR imaging and monitoring can allow precise tracking and measurement of liposomes loaded with markers and therapeutics and/or equivalent cocktails.

The magnetic resonance imaging unit may be arranged to monitor partial oxygen pressure, temperature, pH and/or CO₂ sequentially. However, in the preferred embodiments, they are measured concurrently in real time.

In one preferred embodiment the energy source is an electromagnetic radiation source arranged to operate in the frequency range 1-100 MHz. In another preferred embodiment the energy source is an electromagnetic radiation source arranged to operate in the frequency range 100 MHz to 4 GHz.

An electromagnetic energy source may be a standalone component of the system. However, in some cases it may be possible (and indeed preferable) to use the electromagnetic source which is an integral part of the MRI unit, thereby reducing the number of components in the system and economising on space which is at a premium inside an MRI machine as smaller MRI machines require smaller magnets and are therefore less expensive to buy, to maintain and to operate.

In alternative embodiments, the energy source is an ultrasound unit arranged to operate in the frequency range 20 kHz to 10 GHz. Such embodiments may be particularly advantageous where ultrasound is also to be used as a further treatment modality either for inducing cavitation of naturally occurring or added microbubbles or for releasing liposomally encapsulated therapeutic agents in the region of interest. Again, combining the energy source with the further treatment modality economises on cost and space in the system.

The energy source can be a dual ultrasound transducer unit enabling to transmit a low ultrasound frequency and a high ultrasound frequency. The unit can transmit the frequencies simultaneously or in sequence. The low frequency range is preferably in the range 20 kHz to 1 MHz and the high frequency range is preferably in the range 100 kHz-5 MHz to induce hyperthermia, although higher frequencies up to 10 GHz could be used.

The system preferably further comprises a computation unit for processing the MR data and producing pO₂, temperature, pH and/or CO₂ data. The computation unit may be integral to the MR unit or it may be a separate system component.

Preferably the computation unit is programmed with algorithms (which could be software or hardware, but are preferably software) for carrying out conversion of MR parameter data to pO₂, temperature, pH and/or CO₂ data.

More preferably, the computation unit is connected to the energy source or further treatment modality so as to be able to control the energy source or further treatment modality. By using the calculated data as a feedback mechanism connected to the energy source or treatment modality, better control of the treatment can be carried out. For example, the focus of the energy source can be monitored by spatially detecting temperature increases. If the spatially detected increases are not sufficiently coincident with the region of interest, the direction of the energy source can be corrected. Similarly, if the temperature increases are not high enough or are too high, the focus and/or intensity of the energy source can be adjusted to increase or decrease the hyperthermia. The direction and/or focus of a further treatment modality can also be adjusted or corrected in a similar manner.

According to another aspect, the invention provides software for processing MR parameter data to calculate data for at least one physical parameter related to oxygenation (for example pO₂, temperature, pH and CO₂) and using the calculated data to control an energy source for hyperthermia and/or a further treatment modality.

The software may be loaded directly onto the computation unit or it may be provided in the form of computer executable instructions on a carrier medium such as a compact disc or a floppy disc or a hard disc.

According to another aspect, the invention provides a method for treatment of cancer in a region of interest in a human or animal body comprising the steps of: heating the region of interest by applying a focused energy source; and spatially monitoring at least one physical parameter related to oxygenation (for example temperature, partial oxygen pressure, acidity and carbon dioxide level) within the region of interest using a magnetic resonance imaging unit.

All of the preferred aspects which have been described above in relation to an apparatus and/or system also apply to the corresponding method and software.

This invention represents a system which will provide sufficient selective toxicity to both kill cancer stem cells and cells of the hypoxic fraction of the tumor.

Although in the above description, the term “liposome” has been used, it will be understood that the invention is not limited to the use of liposomes for drug delivery. Other methods of drug delivery, including polymer coated drugs or large molecules with a drug attached to them may equally well be used. Such particles may be either micro- or nano-sized. Accordingly, in this specification, the term “liposome” should be taken to include for example nanoparticle sized liposomes, polymer or lipid nanoparticles, nanospheres, dendrimers and conjugated agents consisting of polymer-linked or pegylated agents.

Similarly, it will be clear that the term “agent” should be taken to include a single drug or a cocktail of pharmaceutical substances.

Preferred embodiments of the invention will now be described, by way of example only, and with reference to the accompanying drawings in which:

FIG. 1 schematically shows the composition of solid tumors,

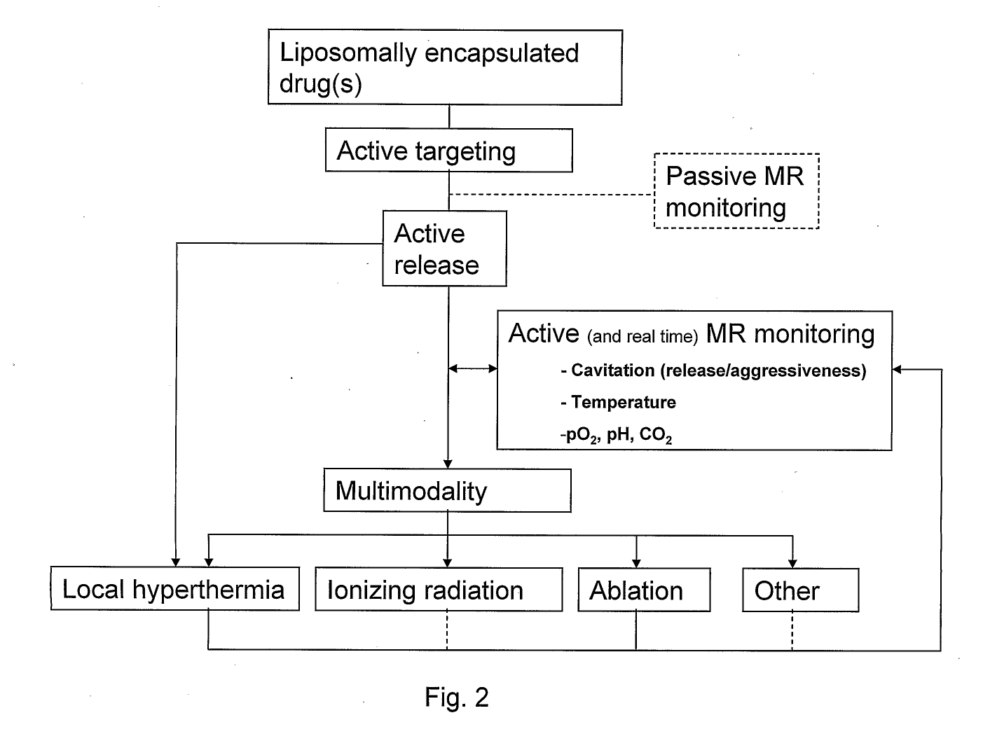

FIG. 2 schematically shows the structure of an MR monitored drug release system,

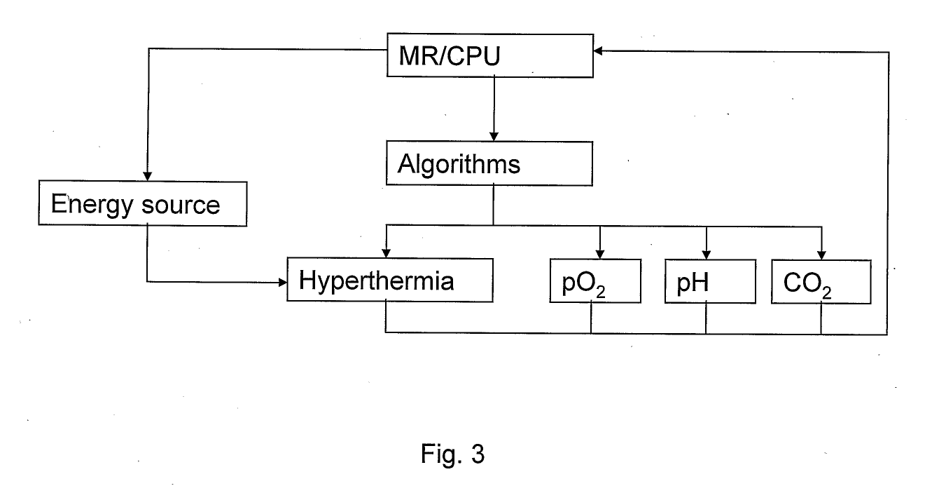

FIG. 3 schematically shows a system for MR monitoring of temperature, pO₂, pH and/or CO₂ comprising an energy source; and

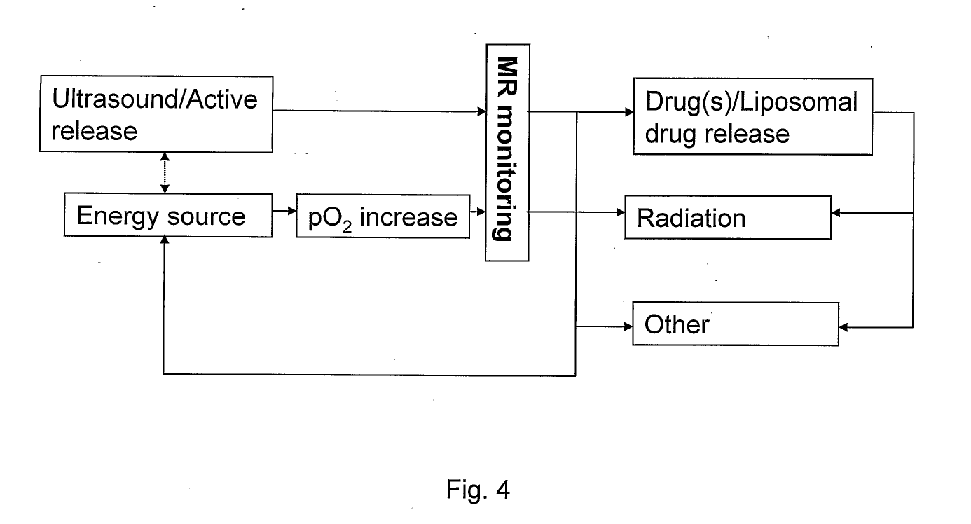

FIG. 4 schematically shows a system for MR Monitoring and multimodal cancer treatment (pH and CO₂ monitoring are not shown).

FIG. 1 shows the composition of solid tumors. Solid tumors constitute two basic components; parenchyma (50-60% neoplastic cells) and stroma. Cancer stem cells make up less than 1% of the cells in a tumor. Stroma is composed of vasculature (1-10%), interstitium and intercellular matrix (30-40%). These proportions are illustrated in the figure in the form of a pie chart. The collagen rich matrix represents the connective tissue that provides both nutritional and structural support for the tumor and its growth. Macroscopically the tumor is composed of a well vascularized outer layer, a hypoxic intermediate fraction and a necrotic core.

FIG. 2 shows the structure of an MR guided drug release system. The primary objective of such a drug delivery system is to provide sufficient selective toxicity to kill both cancer stem cells (CSC) and cells of the hypoxic fraction of the tumor, subject to systemically acceptable and non-hazardous toxic levels. A corollary of this is that such a system will have to fulfill several requirements related to selectivity and multimodality.

Drug accumulation occurs in tumor and/or is selectively activated (by targetting a specific region of interest). Local therapeutic aggressiveness is enhanced. There is synergism between different treatment modalities and quality assurance means monitor drug release and the accumulation of drugs within the tissue in real time by measuring cavitational activity. Further quality assurances are provided by real time monitoring of hyperthermic effects and pO₂, pH and/or CO₂.

The tumor is actively targeted with the use of liposomally encapsulated drugs. Liposomes in general were introduced either to increase the drug concentration in tumor cells and/or to decrease the exposure in normal tissues, exploiting the EPR effect. Most known liposome formulations contain a specific phospholipid, phosphatidyl-ethanolamine (PE), which undergoes a transition from lamellar to inverted micelles structures at low pH and allows fusion of liposomal and endosomal membranes and by consequence destabilization of the endosomes. Therefore, liposomes made of PE are able to release their contents in response to acidic pH within the endosomal system while remaining stable in plasma, thus improving the cytoplasmic delivery of oligonucleotides after endocytosis.

To release the encapsulated drugs, a drug release mechanism, encompassing ultrasound mediated cavitation is used. This accomplishes aggressive liposomal decapsulation and enhances cell membrane permeability. Cavitation involves the nucleation, growth and oscillation of gaseous cavities. Ultrasound can cause stable oscillation or acoustic streaming of naturally occurring or added microbubbles within bodily fluids, or cause the bubbles to collapse, both effects causing shear stress and the rupture of liposomes or polymers, thus releasing the encapsulated therapeutic substances. Selective drug release is achieved by mapping (modelling) of the tumor (region of interest) and then focusing the ultrasound unit so as to induce cavitation only in the well defined tumor region. The ultrasound frequency can be in the range 20 kHz-1 GHz.

Although the role of hyperthermia as a single cancer treatment modality may be limited, there is extensive pre-clinical data showing that in combination with radiation, it represents an extremely potent radiation sensitizer. Concomitant chemotherapy and radiation can be applied to severely hypoxic and resistant tumors. In this respect one can utilize cytotoxic substances like paclitaxel, 5-fluorouracil and hydroxyurea, which are agents with additional radiation sensitizing effects. Further, hyperthermia increases cytotoxicity of various antineoplastic agents. The combined therapy acts synergisticly due to hyperthermia enhanced drug-induced apoptosis. The drugs may be multi-operable, e.g. cytotoxic as well as radiation sensitizing.

The ultrasound induced cavitational effects have to be balanced between energy levels causing liposomal rupture, enhancing cellular permeability, and the prevention of tissue disintegration and the possibility of subsequent proliferation of cancer (stem) cells into the blood stream.

Within the treatment system, hyperthermia can be induced by the drug decapsulating ultrasound unit, by a separate ultrasound transducer or by electromagnetic radiation in both the radiofrequency (RF) (1-100 MHz) and microwave (100 MHz-4 GHz) ranges. The RF coil(s) can be a separate unit Or they can be the built in units within the MR machine itself.

Real time MRI monitoring means related to cavitation, hyperthermia (40-43° C.), pO₂, pH and/or CO₂ facilitates approximate real time concomitant treatment options, including various combinations of drug therapy, hyperthermia, ionizing and/or particle radiation, ablation (55-80° C.) and other treatment options, before or after surgery, with optimization capabilities.

Non-invasive MR temperature imaging is based on the change of various parameters of the MR measurements, namely T1 and T2 (relaxation time), PRF (proton resonance frequency), proton resonance frequency shift, phase changes or D (diffusion coefficient). In Olsrud J. et al. 1998 Phys. Med. Biol. 43 2597-2613, a proton resonance frequency shift MRI thermometry method was outlined. In Ong J. T. et al. 2003 Ohys. Med. Biol. 48 1917-1931, an equivalent sliding window dual gradient echo method was described.

This embodiment of the invention generates localized heat within a living creature, monitors the temperature increase and the subsequent (consequent) increase in oxygenation (e.g. by monitoring partial oxygen pressure (pO₂), pH and/or CO₂) within the targeted volume (region of interest) of the creature. A detected increase in oxygenation levels can be an indicator of successful hyperthermic treatment and/or that the targeted volume is more receptive to particle or ionizing radiation treatment and/or drug treatment with or without subsequent ultrasound exposure.

The pO₂ level may be obtained by measuring the relaxation rate R1 and comparing this with a predetermined relationship between R1 and pO₂.

The primary application of the system is cancer treatment of solid tumors (primary tumors and/or metastases), but natural embodiments can be the treatment to all types of cancers, including lymphoma and leukemia.

FIG. 3 schematically shows another embodiment of the invention. In this embodiment, The system includes a computer (CPU) which is arranged to execute algorithms which provide temperature data in the 30-100 degrees C. range and partial oxygen pressure data in the range 0 mm Hg to 2000 mm Hg, from measurements of various parameters (e.g. T1, T2, PRF, Diffusion) by the MR unit in a defined volume within a living creature. The computer can also provide data on pH and CO₂. The computer/CPU can be an integral part of the MR unit (MR units typically include substantial computing power for complex processing and analysing of large quantities of data to produce MR images) or it can be part of a separate computation unit connected to the MR unit and receiving data from the MR unit. The computer/CPU can control the energy source, the MR unit and/or the other treatment modalities.

The computer is programmed with software algorithms for converting measured parameter data from the MR unit into calculated pO₂, temperature, pH and/or CO₂ data. With sufficient computing power, these calculations can be carried out in real time. The computer uses this calculated data for quality control/feedback relating to the treatment. By comparing the calculated data with prestored mapping data of the region of interest (which has been previously obtained through further MR or CT scans), the computer can determine both spatially and temporally the effectiveness of the treatment. For example, the computer can determine if the applied hyperthermia is sufficiently coincident with the region of interest and it can evaluate how long it takes for the hyperthermia to reach a desired level. The computer can use this analysis for feedback and control of the energy source to correct the focus, direction and/or intensity of the applied hyperthermia. The computer can also use the analysis for feedback and control of other applied treatment modalities such as radiotherapy and/or chemotherapy. For example, the computer can spatially and temporally monitor the pO₂ level within the region of interest and can use that data to begin application of radiotherapy or chemotherapy when a given pO₂ level has been reached, e.g. when the tissue has reached a state of being more receptive to those treatments by virtue of increased oxygenation and increased drug transport to the region through increased blood flow. The computer can also spatially determine where for example the pO₂ level has increased to the desired level and where it has not. The direction and focus of the radiotherapy and/or chemotherapy can then be altered so as to target those areas where the treatment will be effective, without applying the toxic treatments to regions which will not benefit from that treatment.

The measurements of pO₂, temperature, pH and CO₂ are as discussed above in relation to the first embodiment.

The temperature, partial oxygen pressure, pH and/or CO₂ level monitoring can be concurrent, but this is not necessary. The energy source, providing energy and consequently causing a temperature increase into a well defined area, point or volume within a living creature can be the drug release ultrasonic transducer or it can be a separate unit. The energy source can be an ultrasound transducer in the frequency range 20 kHz to 10 GHz. Alternatively the energy source can be an electromagnetic radiation unit operating in the radio frequency (RF) range 1-100 MHz or it can represent electromagnetic radiation unit operating in the microwave range 100 MHz-4 GHz. In this embodiment, the energy source is controlled by the CPU.

This embodiment can be a part of a drug delivery system, encompassing drugs, liposomally encapsulated drugs, and/or active monitoring of cavitation by ultrasound or MR.

In a further embodiment, the features of the first and second embodiments are combined.

The partial oxygen pressure, temperature, pH and/or CO₂ can be determined by graphical comparison of at least two independent measurements of sequences of combinations of relaxation times (T1 and T2), proton resonance frequency shift, phase changes and diffusion coefficient with predetermined relations of sequences of combinations of relaxation times (T1 and T2), proton resonance frequency shift, phase changes and diffusion coefficient to partial oxygen pressure, temperature, pH and/or CO₂.

Further, the partial oxygen pressure, temperature, pH and/or CO₂ can be determined by solving simultaneous equations which are based on the predetermined relations of combinations of relaxation times (T1 and T2), proton resonance frequency shift, phase changes and diffusion coefficient to partial oxygen pressure, temperature, pH and/or CO₂.

FIG. 4 schematically outlines a multimodal treatment system linking the application of active release and/or energy source, increase or changes in pO₂, pH and/or CO₂, subsequent MR monitoring and additional treatment options.

As in the previous embodiments, the energy source targets the region of interest to induce hyperthermia in the region of interest, leading to an increase in oxygenation. Additionally, ultrasound is applied to the region of interest for active drug release (i.e. releasing liposomally encapsulated drugs). As described above, the ultrasound unit may also be used as the energy source for inducing hyperthermia. The MR unit monitors increases and/or changes in the oxygenation level in the region of interest and also monitors the amount of active drug release and drug uptake by monitoring cavitation in the region of interest.

The data obtained by the MR unit relating to oxygenation level and drug uptake is used to control the application of further treatment modalities, such as further drug release, radiation treatment or other treatment modalities (such as ablation). The output of the MR unit is also used as feedback to control the energy source and/or the ultrasound transducer to adjust the direction, focus and/or intensity if necessary so as to achieve the desired levels of hyperthermia and drug release/uptake.

Additionally, the application of further drug release can be used as a further control on the application of radiation treatment or other treatment modalities. For example, the radiation treatment may be controlled according to the amount of radiation sensitizing drug applied.

1. System for treatment of cancer, comprising:
a focusable energy source for targeting a region of interest in a human or animal body to achieve hyperthermia in the region of interest; and
a magnetic resonance imaging unit arranged to monitor at least one physical parameter related to oxygenation level spatially in and around the region of interest.

2. System as claimed in claim 1, wherein the parameter related to oxygenation level is selected from the group of partial oxygen pressure (pθ₂), temperature, acidity (pH) and/or carbon dioxide (CO₂).

3. System as claimed in claim 1, further comprising a further treatment modality operatively connected to the magnetic resonance imaging unit.

4. System as claimed in claim 3, wherein the apparatus is arranged to begin treatment via the further treatment modality when the partial oxygen pressure, pH and/or CO₂ reaches a threshold value.

5. System as claimed in claim 3, wherein the apparatus is arranged to control the focus of the further treatment modality based on the measurements taken by the magnetic resonance imaging unit.

6. System as claimed in claim 3, wherein the further treatment modality is a radiation unit.

7. System as claimed in claim 3, wherein the further treatment modality is an ultrasound unit arranged to induce cavitation in the region of interest.

8. System as claimed in claim 7, wherein the ultrasound unit is arranged to induce cavitation which activates a therapeutic agent in the region of interest.

9. System as claimed in claim 1, wherein the magnetic resonance imaging unit is arranged to monitor more than one of partial oxygen pressure, temperature, pH and CO₂ level concurrently in real time.

10. System as claimed in claim 1, wherein the energy source is an electromagnetic radiation source arranged to operate in the frequency range 1-100 MHz.

11. System as claimed in claim 1, wherein the energy source is an electromagnetic radiation source arranged to operate in the frequency range 100 MHz to 4 GHz.

12. System as claimed in claim 10, wherein the electromagnetic energy source is part of the magnetic resonance imaging unit.

13. System as claimed in claim 1, wherein the energy source is an ultrasound unit arranged to operate in the frequency range 20 kHz to 10 GHz.

14. System as claimed in claim 1, wherein the magnetic resonance imaging unit is arranged to measure combinations of relaxation times, proton resonance frequency, phase changes and diffusion coefficient and to relate these measurements to predetermined relations between those parameters and partial oxygen pressure, temperature, pH and/or CO₂.

15. System as claimed in claim 1, wherein the partial oxygen pressure is determined by graphical comparison of at least two independent measurements of sequences of combinations of relaxation times (T1 and T2), proton resonance frequency shift, phase changes and diffusion coefficient with predetermined relations of sequences of combinations of relaxation times (T1 and T2), proton resonance frequency shift, phase changes and diffusion coefficient to partial oxygen pressure.

16. System as claimed in claim 1, wherein the temperature is determined by a graphical comparison of at least two independent measurements of sequences of combinations of relaxation times (T1 and T2), proton resonance frequency shift, phase changes and diffusion coefficient with predetermined relations of sequences of combinations of relaxation times (T1 and T2), proton resonance frequency shift, phase changes and diffusion coefficient to temperature.

17. System as claimed in claim 1, wherein the partial oxygen pressure is determined by solving simultaneous equations which are based on the predetermined relations of combinations of relaxation times (T1 and T2), proton resonance frequency shift, phase changes and diffusion coefficient to partial oxygen pressure.

18. System as claimed claim 1, wherein the temperature is determined by solving simultaneous equations which are based on the predetermined relations of combinations of relaxation times (T1 and T2), proton resonance frequency shift, phase changes and diffusion coefficient to temperature.

19. System as claimed in claim 1, further comprising a computation unit for processing MR data and producing pθ₂, temperature, pH and/or CO₂ data.

20. System as claimed in claim 19, wherein the computation unit is integral to the MR unit.

21. System as claimed in claim 19, wherein the computation unit is programmed with algorithms for carrying out conversion of MR parameter data to pθ₂, temperature, pH and/or CO₂ data.

22. System as claimed in claim 21, wherein the algorithms are software algorithms.

23. System as claimed in claim 19, wherein the computation unit is connected to the energy source or further treatment modality so as to be able to control the energy source or further treatment modality.

24. Method for treatment of cancer in a region of interest in a human or animal body comprising the steps of: heating the region of interest by applying a focused energy source; and spatially monitoring at least one physical parameter related to oxygenation level within the region of interest using a magnetic resonance imaging unit.

25. Method as claimed in claim 24, wherein the parameter related to oxygenation level is selected from the group of temperature, partial oxygen pressure, acidity and carbon dioxide level.

26. Method as claimed in claim 24, further comprising the step of controlling a further treatment modality based on the measurements taken by the magnetic resonance imaging unit.

27. Method as claimed in claim 26, wherein treatment by the treatment modality is begun when the partial oxygen pressure, pH and/or CO₂ level has reached a threshold value.

28. Method as claimed in claim 26, wherein the focus of the treatment modality is controlled based on the measurements taken by the magnetic resonance imaging unit.

29. Method as claimed in claim 26, wherein the further treatment modality is a radiation unit;

30. Method as claimed in claim 26, wherein the further treatment modality is an ultrasound unit inducing cavitation in the region of interest.

31. Method as claimed in claim 30, wherein the ultrasound unit induces cavitation which activates a therapeutic agent in the region of interest.

32. Method as claimed in claim 24, wherein the magnetic resonance imaging unit monitors more than one of partial oxygen pressure, temperature, pH and/or CO₂ concurrently in real time.

33. Method as claimed in claim 24, wherein the energy source emits electromagnetic radiation in the frequency range 1-100 MHz.

34. Method as claimed in claim 24, wherein the energy source emits electromagnetic radiation in the frequency range 100 MHz to 4 GHz.

35. Method as claimed in claim 33, wherein the magnetic resonance imaging unit functions as the electromagnetic energy source.

36. Method as claimed in claim 24, wherein the energy source emits ultrasound in the frequency range 20 kHz to 10 GHz.

37. Software for processing MR parameter data to calculate data for at least one physical parameter related to oxygenation and using the calculated data to control an energy source for hyperthermia and/or a further treatment modality.
